(19) United States
(12) Patent Application Publication
(10) Pub. No.: US 2020/0188286 A1
(43) Pub. Date: Jun. 18, 2020

(54) CLEANSING HAIR OIL, METHOD FOR APPLYING SAME, AND USE OF SAME FOR CARE OF KERATIN FIBERS

(71) Applicant: Henkel AG & Co. KGaA, Duesseldorf (DE)

(72) Inventors: JENS DELOWSKY, Norderstedt (DE); SYLVIA KERL, Boenningstedt (DE)

(73) Assignee: Henkel AG & Co. KGaA, Duesseldorf (DE)

(21) Appl. No.: 16/689,975

(22) Filed: Nov. 20, 2019

(30) Foreign Application Priority Data
Dec. 18, 2018 (DE) 10 2018 222 097.2

(51) Int. Cl. A61K 8/92

(52) U.S. Cl. CPC A61K 8/922; A61Q 5/06

(57) ABSTRACT

The present disclosure relates to an anhydrous hair oil which comprises a cosmetic oil and an emulsifier. The present disclosure also relates to a method which comprises a step in which a hair oil is applied to keratin fibers, in particular human hair, and which comprises a subsequent step in which the hair oil is rinsed out.

### CROSS-REFERENCE TO RELATED APPLICATION

This application claims priority to German Patent Application No. 10 2018 222 097.2, filed Dec. 18, 2018, which is incorporated herein by reference in its entirety.

### TECHNICAL FIELD

Hair oils have long been considered by users thereof to be a simple, efficient and usually economical way of providing effective care for hair. Which oils are suitable and the way in which they act have been the topic of much discussion among users. Hair oils have been a popular care product for centuries, for example in North Africa and in many other areas of the world, in particular where there is much sunlight. In Germany, where there is less sunlight, hair oil traditions started for example in Victorian times and then disappeared again to a large extent. However, hair oils have once again become increasingly fashionable in recent years. Whereas certain plant oils were previously a secret ingredient so to speak, a large number of providers have recently introduced their own hair oil variants onto the market.

### BACKGROUND

This trend seems to have come about since an increasing number of users prefer products containing the fewest possible, manageable number of ingredients, for example since it is suspected that certain chemical-synthetically produced ingredients have unwelcome properties for humans or the environment. For example, more silicones than oils are actually contained in some hair oils advertised as being natural. The silicones make the hair shiny, easily combed, and leave it feeling soft. However, critics assert that these effects are created since the silicones seal the hair, and do not provide a care effect. On the whole, the question is whether synthetic or natural ingredients are preferred by each individual user, with both options offering their own advantages and disadvantages.

Hair oils are used traditionally to provide care, wherein they either are applied independently of hair washing or are applied for a certain period of time of up to a few hours prior to hair washing and are then washed out using a shampoo. The present disclosure described here, by contrast, in accordance with a first aspect relates to a cleansing hair oil. In accordance with a second aspect the present disclosure relates to a method for applying the cleansing hair oil to keratin fibers, in particular human hair, and in accordance with a third aspect the present disclosure relates to the use of the cleansing hair oil for the care of keratin fibers, in particular human hair. In the following description, for the sake of simplicity for example, effects will be described sometimes only in respect of hair or only in respect of human hair, although they are also attainable for other keratin fibers.

Conventional hair oils are versatile to a certain extent in respect of their use. For example, they can be used as a treatment before hair is washed. To this end the corresponding oil is distributed sparingly in the hair, in particular on the ends of the hair, for example thirty to sixty minutes prior to hair washing. Hair oil can also be used optionally as a “leave-in” product. To this end a small portion of hair oil is distributed over the ends after hair washing. The oil also protects the hair against heat when blow drying or straightening. Applied to wet hair after washing, hair oil ensures easier combing. To this end it seals the hair, which makes it shiny, and prevents against splitting. An important factor for hair oils that are intended for use as a leave-in product is that they should not unnecessarily weigh down the hair. Good hair oils do not have an excessively weighty or greasy effect on the hair, provided the dosing is correct, however, these properties cannot be completely avoided. A useful secondary aspect of the use of hair oil is that the hair is not only cared for and made shiny, but, depending on the oil, the hair also dries more quickly than would otherwise be the case.

As can be concluded, however, from the above explanation, in the case of conventional hair oils either a certain greasiness of the hair after application must always be expected as an inherent feature of the system, or said oils must be used in combination with a shampoo, that is to say must be washed out with shampoo after a certain reaction time. In other words conventional hair oils do not have any cleansing properties and are greasy. This property is perceived to be disadvantageous by some users since a greasy appearance of the hair is considered to be undesirable. The fact that some of the care effect of an oil is neutralized again by the subsequent application of a shampoo in order to avoid greasiness is perceived to be disadvantageous, in particular by users having relatively dry hair.

The inventors of the embodiments claimed herein confronted the problem of providing a hair oil that offers the user both cleansing and caring properties, and also enabling a method in which the hair oil is used, and a use of the hair oil. This problem is the problem forming the basis of the present disclosure. The problem is solved by a first aspect of the present disclosure.

### BRIEF SUMMARY

A hair oil and methods using the hair oil are provided herein. An exemplary hair oil includes a cosmetic oil and an emulsifier. An exemplary method includes the steps of applying such a hair oil to keratin fibers and rinsing out the hair oil. In another embodiment, an exemplary method includes the steps of applying such a hair oil according to human hair, leaving the hair oil on the human hair for a period of time of from about 10 seconds to about 30 minutes, and rinsing out the hair oil from the human hair.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

In accordance with the first aspect an anhydrous hair oil comprises a cosmetic oil and an emulsifier. Emulsifiers have not been used previously in hair oils. The hair oils in the sense of the present disclosure are anhydrous. Here, anhydrous means, for the purposes of the present disclosure, that no water has been added intentionally. Nevertheless, minimal water amounts contained in the starting substances or introduced unintentionally during a production process can be present, for example at most about 1% by weight, more preferably at most about 0.3% by weight, particularly preferably at most about 0.1% by weight, in relation to the total hair oil.

The cosmetic oil is not particularly limited and can be an oil or a mixture usually used in the cosmetics field. In particular, it can comprise a natural oil, a silicone oil, an ester oil or another oil suitable for cosmetic purposes. A natural oil as contemplated herein described here is an oil that is found in naturally occurring raw material sources, for example of microbial, plant or animal origin, but in particular plant, origin and can be obtained therefrom. Examples of a natural oil of this kind are apricot kernel oil, avocado oil, macadamia nut oil, argan oil, coconut oil, jojoba oil and castor oil. However, it can also be an oil obtained from such oils by simple method steps which change only slightly, such as a neutral oil, also referred to as MCT oil (medium chain triglyceride oil), that is to say a mixture of triglycerides of natural origin with medium-chain fatty acids. The starting basis for such an oil is generally provided by palm kernel oil or coconut oil. Neutral oil is irritant-free, easily applied, and rapidly absorbed. An example of another suitable oil is dioctyl carbonate, commercially obtainable as Cetiol® CC. Each of the usable oils is associated with its own advantages or disadvantages. For example, castor oil is associated with the following advantageous effects: the hair and the hair root are strengthened, hair growth is encouraged, unmanageable and dry hair is made smooth and soft, the hair is provided with a healthy shine, and the oil penetrates deep into the inner layers of the scalp, which is associated with natural protection of the scalp.

The emulsifier is not particularly limited, provided it is suitable for cosmetic purposes. For example, the emulsifier comprises ethoxylated fatty acids, ethoxylated or non-ethoxylated sorbitol esters, PEG derivatives based on glycerol or glycol, non-ethoxylated glycerol or glycol derivatives, or a mixture thereof The advantage of the use of such an emulsifier in a hair oil lies in the fact that this oil emulsifies by itself with water, in particular warm water, on the hair and on the scalp, and therefore it is also referred to as self-emulsifying, whereby any oil present in excess can be removed simply by being rinsed out, without the need for a shampoo. Nevertheless, there is no greasy feeling of the skin or hair after application. A care effect is achieved as a result, however, the hair is not weighed down or made greasy and does not have to be loaded additionally by the use of a shampoo, whereby people with dry hair benefit in particular. A specific one of the usable emulsifiers if propylene glycol dodecyl ether, which is commercially obtainable as Brij® L4.

In the case of the hair oil according to the first aspect the cosmetic oil preferably accounts for from about 80% by weight to about 97% by weight, more preferably from about 84% by weight to about 95% by weight, particularly preferably from about 88% by weight to about 93% by weight of the hair oil. In these ranges an emulsifiability sufficient for rinsing out with water is attained increasingly, however, pronounced caring properties are also still ensured.

In addition, the emulsifier preferably accounts for from about 3% by weight to about 20% by weight, more preferably from about 4% by weight to about 15% by weight, particularly preferably from about 5% by weight to about 10% by weight of the hair oil. In these ranges an emulsifiability sufficient for rinsing out with water is attained increasingly, however, pronounced caring properties are also still ensured.

In addition the castor oil preferably accounts for from about 0,1% by weight to about 10% by weight, preferably from about 0.3% by weight to about 6% by weight, particularly preferably from about 0.7% by weight to about 3% by weight of the hair oil. In these ranges it is ensured increasingly that both the above-described particular advantageous properties of the castor oil take effect, and also that the hair oil can still contain a sufficient proportion of a further oil, so that it can also have the advantageous effects, which are associated with the further oil.

The hair oil as contemplated herein preferably comprises one of the following combinations of oil and emulsifier: a silicone oil with ethoxylated fatty acids, an ester oil with ethoxylated fatty acids, another oil suitable for cosmetic purposes with ethoxylated fatty acids, apricot kernel oil with ethoxylated fatty acids, avocado oil with ethoxylated fatty acids, macadamia nut oil with ethoxylated fatty acids, argan oil with ethoxylated fatty acids, coconut oil with ethoxylated fatty acids, jojoba oil with ethoxylated fatty acids, castor oil with ethoxylated fatty acids, neutral oil with ethoxylated fatty acids; a silicone oil with ethoxylated or non-ethoxylated sorbitol esters, an ester oil with ethoxylated or non-ethoxylated sorbitol esters, another oil suitable for cosmetic purposes with ethoxylated or non-ethoxylated sorbitol esters, apricot kernel oil with ethoxylated or non-ethoxylated sorbitol esters, avocado oil with ethoxylated or non-ethoxylated sorbitol esters, macadamia nut oil with ethoxylated or non-ethoxylated sorbitol esters, argan oil with ethoxylated or non-ethoxylated sorbitol esters, coconut oil with ethoxylated or non-ethoxylated sorbitol esters, jojoba oil with ethoxylated or non-ethoxylated sorbitol esters, castor oil with ethoxylated or non-ethoxylated sorbitol esters, neutral oil with ethoxylated or non-ethoxylated sorbitol esters; a silicone oil with PEG derivatives based on glycerol or glycol, an ester oil with PEG derivatives based on glycerol or glycol, another oil suitable for cosmetic purposes with PEG derivatives based on glycerol or glycol, apricot kernel oil with PEG derivatives based on glycerol or glycol, avocado oil with PEG derivatives based on glycerol or glycol, macadamia nut oil with PEG derivatives based on glycerol or glycol, argan oil with PEG derivatives based on glycerol or glycol, coconut oil with PEG derivatives based on glycerol or glycol, jojoba oil with PEG derivatives based on glycerol or glycol, castor oil with PEG derivatives based on glycerol or glycol, neutral oil with PEG derivatives based on glycerol or glycol; a silicone oil with non-ethoxylated glycerol or glycol derivatives, an ester oil with non-ethoxylated glycerol or glycol derivatives, another oil suitable for cosmetic purposes with non-ethoxylated glycerol or glycol derivatives, apricot kernel oil with non-ethoxylated glycerol or glycol derivatives, avocado oil with non-ethoxylated glycerol or glycol derivatives, macadamia nut oil with non-ethoxylated glycerol or glycol derivatives, argan oil with non-ethoxylated glycerol or glycol derivatives, coconut oil with non-ethoxylated glycerol or glycol derivatives, jojoba oil with non-ethoxylated glycerol or glycol derivatives, castor oil with non-ethoxylated glycerol or glycol derivatives, neutral oil with non-ethoxylated glycerol or glycol derivatives.

The hair oil as contemplated herein can contain a perfume; an example of a perfume is the commercially obtainable product “Parfum After Beauty 801628”. The proportion of the perfume in the hair oil accounts for example for about 0.3% by weight.

The problem forming the basis of the present disclosure described here is also solved by a second aspect. Features described for the first aspect can also be found in this and further aspects, provided there is no specific impediment. The second aspect of the present disclosure described herein relates to a method that comprises a step in which a hair oil in accordance with the first aspect is applied to keratin fibers, in particular human hair, and that comprises a subsequent step in which the hair oil is rinsed out. The attainable advantages correspond to those described in relation to the first aspect.

The method preferably comprises a further step, in which the hair oil is left on the keratin fibers, in particular the human hair, for a period of time from about 10 seconds to about 30 minutes, preferably from about 1 minute to about 10 minutes, particularly preferably from about 3 minutes to about 10 minutes prior to rinsing out the hair oil. The previously described, advantageous effects are attained increasingly as a result of this approach.

The problem addressed by the present disclosure described here is also solved by a third aspect. This third aspect of the present disclosure described here relates to a use, wherein the hair oil according to a first aspect is used to care for keratin fibers, in particular human hair.

A specific example of the first aspect of the present disclosure is a hair oil having a composition defined in Table 1 below. The proportions specified in the table relate to the total weight of the hair oil.

TABLE 1

| Ingredient | Proportion in % by weight |
|---|---|
| Caprylic/Capric Triglyceride (INCI) | 48.70 |
| Laureth-4 (INCI)₍₁₎ | 8.00 |
| Dicaprylyl Carbonate (INCI)₍₂₎ | 42.00 |
| Parfum Alter Beauty 801628 | 0.30 |
| Castor oil | 1.00 |

₍₁₎Commercially obtainable from Sigma-Aldrich under the trade name Brij ® L4
₍₂₎Commercially obtainable from BASF Care Creations under the trade name Cetiol ® CC

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair oil comprising:
a cosmetic oil; and
an emulsifier.

2. The hair oil according to claim 1, wherein the cosmetic oil comprises at least one of a natural oil, a silicone oil and an ester oil.

3. The hair oil according to claim 1, wherein the cosmetic oil comprises castor oil.

4. The hair oil according to claim 1, wherein the emulsifier comprises an ethoxylated fatty acid.

5. The hair oil according to claim 1, wherein the cosmetic oil accounts for from about 80% by weight to about 97% by weight of the hair oil.

6. The hair oil according to claim 1, wherein the emulsifier accounts for from about 3% by weight to about 20% by weight of the hair oil.

7. The hair oil according to claim 3, wherein the castor oil accounts for from about 0.1% by weight to about 10% by weight of the hair oil.

8. A method, comprising the steps of:
applying a hair oil according to claim 1 to keratin fibers, and
rinsing out the hair oil.

9. The method according to claim 8, comprising the step:
leaving the hair oil on the keratin fibers for a period of time of from about 10 seconds to about 30 minutes before rinsing out the hair oil.

10. A method, comprising the steps of:
applying a hair oil according to claim 1 to human hair,
leaving the hair oil on the human hair for a period of time of from about 10 seconds to about 30 minutes, and
rinsing out the hair oil from the human hair.
